(19) United States
(12) Patent Application Publication
(10) Pub. No.: US 2022/0111082 A1
(43) Pub. Date: Apr. 14, 2022

(54) TERMINAL STERILIZATION OF BIOLOGICS

(71) Applicant: Trevor Percival Castor, Arlington, MA (US)

(72) Inventor: Trevor Percival Castor, Arlington, MA (US)

(73) Assignee: Trevor P. Castor, Woburn, MA (US)

(21) Appl. No.: 17/499,836

(22) Filed: Oct. 12, 2021

Related U.S. Application Data
(60) Provisional application No. 63/090,713, filed on Oct. 12, 2020.

(51) Int. Cl. A61L 2/00; B01F 5/04

(52) U.S. Cl. CPC A61L 2/0088; B01F 2215/0073; A61L 2202/121; B01F 5/0405

(57) ABSTRACT

The invention involves the use of supercritical or near supercritical fluids to inactivate pathogens in biologic materials which may or may not be contaminated by pathogens. The pathogen reduced material is then inserted into empty, sterile containment vessels. The apparatus can be used as a means to achieve terminal sterilization of the biologic materials. The preferred method of use for the apparatus includes operation of a conveyor belt to move and fill bottles, flasks, containers, or vials in an assembly line to create the finished product in an effective and timely fashion.

### FIELD OF INVENTION

This invention relates to process and apparatus for inactivation of potentially pathogenic microbes using critical, near critical and/or supercritical fluids as a means to achieve terminal sterilization.

### BACKGROUND OF THE INVENTION

Human exposure to pathogens in many cases can lead to a host of diseases, some of which are fatal. Therefore, sterilization of products, fluids, medications, and biological compounds is a necessary means of eliminating such microbial life forms. Sterilization is the process used to rid materials of microbes and pathogens and can be accomplished by various methods such as the use of heat, chemicals, gas plasma, irradiation, high pressure, filtration, and compounds such as ethylene oxide, peracetic acid, or aqueous gluteraldehyde.

A level of 10⁻⁶ is recommended for a material or compound to reach sterilization on a sterilized device. Terminal sterilization is the final sterilization of instruments, equipment, and materials which thereby renders the materials safe for use and handling.

Embodiments of this invention provide novel methods and apparatus designed to achieve safe and efficient terminal sterilization.

### SUMMARY OF THE INVENTION

Embodiments of the present invention utilize a supercritical, critical, or near critical fluid. A substance becomes a critical fluid at which its conditions are equal to its critical temperature and critical pressure. The parameters of critical temperature and critical pressure are intrinsic thermodynamic properties of stable pure compounds and mixtures.

For example, carbon dioxide becomes a supercritical fluid at conditions which equal or exceed its critical temperature of 31.1° C. and its critical pressure of 72.8 atm (1,070 psig). Once a substance is within the supercritical fluid region, customarily ambient gaseous substances, such as carbon dioxide, become dense phase fluids which have been discovered to exhibit greatly enhanced solvating power. At a pressure of 3,000 psig (204 atm) and a temperature of 40° C., carbon dioxide maintains a density of approximately 0.8 g/cc and behaves similarly to a nonpolar organic solvent, having a dipole moment of zero Debyes. A supercritical fluid displays greater solvation power than a gas or liquid because at the supercritical point a substance has combined properties of both a gas and a liquid.

The process consists of the steps of contacting a material or substance with a critical, near critical or supercritical fluid through the use of 2 in-line streams. The first in-line stream consists of the potentially pathogenic material and the second in-line stream consists of the critical, supercritical, or near critical fluid. Once the material is contacted with the critical, near critical or supercritical fluid, said critical, supercritical or near critical fluid has the ability to be received by a material or substance and upon removal causes inactivation of the pathogen. More specifically, the method comprises the step of removing the critical, supercritical or near critical fluid from the material or substance, thereby, rendering the pathogen inactive.

The term “pathogen” is used herein to describe bacteria, virus, and fungi particles. The term “inactivation” means the pathogens are made unable to replicate or infect a material or substance. The term “product fluid” is used to describe the material fluid or first in-line fluid; this may also be referred to as “material” or “material fluid.” In chemistry, the term “critical fluid” is described as a gas at or above its critical temperature and at or above its critical pressure. The term “near-critical” is used to describe as approaching or being critical. Supercritical fluids may often be referred to by their abbreviation, “SCF,” in this application. The term “terminal containment vessel” is used herein to describe a vial, bottle, syringe, or container that the pathogen-free material will ultimately be placed.

Supercritical, critical or near critical fluids' solvating properties are influenced by modifiers, commonly referred to as cosolvents and entrainers. These modifiers are normally somewhat polar organic solvents such as acetone, ethanol, methanol, methylene chloride or ethyl acetate. Variation in the proportion of a modifier allows for a wide range in variation of the solvent power. At near-critical pressure or higher and to near critical temperature, the solvent may be combined with a multitude of cells to saturate the cells with the solvent under the prescribed conditions.

Supercritical fluids can exhibit density like a liquid yet still retain properties of high diffusivity and low viscosity like a gas. The latter increases rates in which mass can transfer, thereby significantly reducing processing times. Further, supercritical. fluids allow for facile penetration into microporous materials, which results in great efficiency and larger yields than using a liquid or gas alone.

Preferred fluids are those that are gases at ambient temperature and have critical temperatures between 0° and 100° C., but most preferably between 0° and 60° C. to preserve biological activity. A temperature of above 0° C. is desired for aqueous materials to avoid freezing. Preferred fluids include fluorocarbons such as chlorodifluoromethane, alkanes such as ethylene, propane and ethane, and binary fluids such as nitrous oxide and carbon dioxide. These fluids can be used with cosolvents, small quantities of polar entrainers or modifiers. The cosolvents can affect the polarity of the critical fluid, thereby enhancing the capacity of the critical fluid to inactivate the pathogens in certain materials. The present apparatus can be used to substantially reduce and eradicate the viral load of materials.

### BRIEF DESCRIPTION OF THE DRAWINGS

FIG. 1 depicts an apparatus having features of the present invention.

### DETAILED DESCRIPTION

Embodiments of the present invention will be described in detail as a method or apparatus for inactivating pathogens. This detailed description is directed to the best mode or modes to practice the invention as presently contemplated. The methods and apparatus of the present invention feature two in-line streams; one consisting of product fluid and the other consisting of supercritical, near critical or critical fluids.

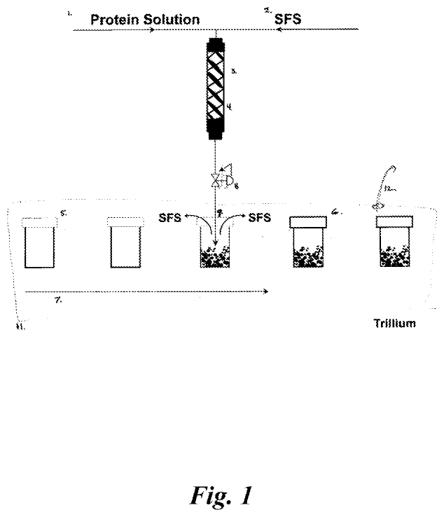

FIG. 1 illustrates a preferred apparatus for inactivating the pathogens through the means of the two-inline streams combining in a third conduit.

The preferred apparatus includes the two in-line fluids combining in a third conduit, where the mixing and extraction process is conducted. The first in-line material solution (1) is combined with the second in-line supercritical fluids (2), in a mixing device, referred to as the third conduit (3). The supercritical fluids work to extract pathogens from the solution or substance (4). Post-extraction the materials are inserted into terminal containment vessels (5) where the material is eventually stored therein (6). This may be done on a conveyer belt, as a preferred apparatus, but is not limited to such an apparatus (7). A backpressure regulator is also included in the apparatus (8). A nozzle (9) for inserting said mixture into containment vessel by means of the belt. To ensure maximum sterilization the containment vessels and belt are enclosed in a device such as a laminar hood or vacuum (11). The enclosed vacuum or hood should also include an aperture to release gases of the admixture (12).

Embodiments of the present method and apparatus are ideally suited for biological materials that need be free of microbes and pathogens. The invention will inactivate microbes and pathogens through the use of the supercritical fluid expansion process by combining the biological material with the solvent. The embodiments of the invention will cause the biological material to be separated from the microbes and pathogens, rendering the biological material free of the microbes and pathogens. Thereby, rendering the biological material sterile and ultimately terminally sterile.

A particular super critical, critical, or near critical fluid will be selected based upon at least two factors. First, the fluid should be capable of achieving the desired result of inactivating viruses. Secondly, the fluid should be selected with the goal of minimally affecting the material being treated. For example, in the case of a protein, a fluid with an operating temperature below 60° C., that does not chemically denature or otherwise adversely affect the material is preferred.

The particular fluid selected as well as the time of exposure of the critical fluid to the material, the temperature and pressure of the mixture are interdependent and together or individually may determine the appropriate conditions for the desired result. Namely, the time the critical fluid is exposed to the material may affect the degree of the pathogen inactivation. Therefore, the conditions for treating the material include sufficient time exposure to ensure that the material has the desired pathogen reduction or complete eradication of the pathogen post-treatment.

Preferably, the first in-line fluid (material) is separated from the second in-line fluid (critical fluid) in the third conduit under aseptic conditions. If for example, a solution containing a protein to achieve separation, the mixture is decompressed thereby resulting in a phase separation of the fluid from the solution containing the proteinaceous product. The material then is isolated under aseptic conditions.

Isolating the material refers to separating the material from the equipment of the invention such as in a sterile bottle, flask, container or vial. It does not simply mean separating the material from the critical fluid into a compartment or container that is part of the sterilization equipment of the invention. Nevertheless, it should be understood that the terminal containment vessel used in isolating the material may be at least temporarily attachable to the equipment of the invention so as to facilitate in the transfer of the material from the apparatus to its isolated state in the terminal containment vessel.

Embodiments of the present apparatus are ideally suited for inactivating pathogens that is associated with biological materials. The supercritical, critical or near critical fluid is selected to have minimal effects on the material aside from the eradication of pathogens.

The preferred apparatus includes a source of fluid, a high pressure, recirculation loop, a separation chamber, and at least one low-pressure trap. Viral inactivation occurs in the high-pressure, recirculation loop, which is rated for continuous operation at 5,000 prig and 100° C.

Thus, the inventions have been described in detail with respect to the best mode. Those skilled in the art will readily understand that the description is capable of modification and alteration without departing from the teaching herein. Therefore, the invention should not be limited to the precise details presented but should encompass the subject matter of the claims that follow and their equivalents.

1. An apparatus for delivering a material in a terminal containment vessel comprising:
(a) a first conduit for conveying a first fluid stream having a material for placing in a containment vessel; (b) a second conduit for conveying a second fluid stream having a supercritical, critical or near critical fluid; (c) a third conduit in communication with said first conduit and said second conduit for forming an admixture fluid of said first fluid and second fluid under super critical, critical, or near critical conditions; and (d) a delivery means for placing said admixture into said containment vessel.

2. The apparatus of claim 1 wherein said delivery means comprises a nozzle, said delivery means includes a backpressure regulator, and said third conduit comprises an in-line mixer.

3. A method of inactivating pathogens potentially associated with a fluid containing a material to be placed in a terminal containment vessel comprising the steps of: (a) producing a first fluid stream of said product fluid; (b) providing a second fluid stream of a supercritical, critical or near critical fluid; (c) forming an admixture fluid of said first fluid and second fluid under supercritical, critical or near critical fluid; and (d) directing said admixture into said terminal containment vessel to deposit said materials in said containment vessel as said supercritical, critical, or near critical fluid is vested to leave a material that is pathogen free.

4. A method of claim 3 for depositing said admixture in containment vessel comprising of the use of a nozzle.

5. A method of claim 3 for conveying the vials comprising of (a) a conveyor belt, turntables, endless belts, slide surfaces; (b) a vial conveying means is enclosed to maintain sterility; (c) a laminar hood or vacuum to achieve enclosure; and (d) an enclosed vial conveying means that includes an aperture to release gases of admixture,

6. A method of claim 3 or sealing the containment vessels by placing covers or lids on top of containment vessels.
